(19) United States
(12) Patent Application Publication
(10) Pub. No.: US 2024/0417696 A1
(43) Pub. Date: Dec. 19, 2024

(54) CELL-DERIVED VESICLE RICH IN PROTEIN HOMEOSTASIS REGULATORS AND METHOD OF PREPARING SAME

(71) Applicant: MDimune Inc., Seoul (KR)

(72) Inventors: Shin Gyu Bae, Gyeonggi-do (KR); Seung Wook Oh, Gyeonggi-do (KR); Dong Woo Han, Gyeonggi-do (KR); Jun Sik Yoon, Seoul (KR); Ji Hye Lee, Incheon (KR)

(21) Appl. No.: 17/927,335

(22) PCT Filed: May 17, 2021
(86) PCT No.: PCT/KR2021/006154

§ 371 Date: Nov. 22, 2022

(30) Foreign Application Priority Data
May 22, 2020 (KR) 10-2020-0061867

(51) Int. Cl. C12N 5/09; A61K 9/16; A61K 9/20; A61K 9/48; A61K 35/13; A61K 35/17; A61K 35/28; C12N 5/0775; C12N 5/0783

(52) U.S. Cl. CPC C12N 5/0693; A61K 9/1623; A61K 9/1652; A61K 9/1664; A61K 9/2013; A61K 9/2018; A61K 9/2059; A61K 9/4825; A61K 9/4858; A61K 9/4866; A61K 35/13; A61K 35/17; A61K 35/28; C12N 5/0646; C12N 5/0667

(57) ABSTRACT

The present disclosure relates to a cell-derived vesicle rich in intracellular protein homeostasis regulators, and to a method of preparing same. The cell-derived vesicle of the present disclosure is characterized by containing an abundance of a specific protein compared to that in a naturally secreted exosome or the originated cell, and by virtue of said characteristic, can be utilized for various purposes such as in a pharmaceutical composition, a diagnostic composition, and a composition for drug delivery. Using the cell-derived vesicle rich in intracellular protein homeostasis regulators and the method of preparing same of the present disclosure, it is possible to easily deliver the intracellular protein homeostasis regulators to a target, such as a cell, thereby easily maintaining protein homeostasis in cells. Accordingly, the present disclosure may also be utilized for “a use for the prevention or treatment of disease caused by a protein homeostasis regulation failure within cells” or “a use for preventing aging”, and thus can be widely used in the drug industry.

### CROSS-REFERENCE TO RELATED APPLICATIONS

This is a U.S. National Phase of International Application PCT/KR2021/006154, filed May 17, 2021, which claims priority to Korean Patent Application No. 10-2020-0061867, filed May 22, 2020.

### TECHNICAL FIELD

The present disclosure relates to a cell-derived vesicle rich in intracellular protein homeostasis regulators and a method of preparing the same, in which the cell-derived vesicle of the present disclosure is characterized by containing an abundance of a specific protein compared to that in a naturally secreted exosome or the originated cell, and due to this characteristic, can be utilized for various purposes such as a pharmaceutical composition, a diagnostic composition, and a composition for drug delivery.

### BACKGROUND ART

Irwin Rose of the United States and Dr. Aaron Ciechanover and Avram Hershko of Israel found a process of selective degradation of a protein to which a molecule called ubiquitin was attached by a ubiquitin-proteasome system (UPS), and then received the Nobel prize for chemistry in 2004. The ubiquitin-proteasome system is involved in biological processes such as removal of defective proteins, cell division, and repair of damaged DNA in eukaryotic cells. In diseases such as degenerative brain disease, when the ubiquitin proteasome system is abnormal, toxic proteins are accumulated in the body. In particular, in Alzheimer's disease, which is a degenerative brain disease, it has been reported that an amyloid protein inhibits the activity of the ubiquitin proteasome system, and the proteasome activity of the brain was reduced in an Alzheimer's disease animal model.

In addition, chaperonin-containing T-complex polypeptide 1 (CCT) is a protein constituting chaperone. The chaperone is a protein that functions in the folding and assembly of a protein conformation. The chaperone prevents aggregation of proteins and adjusts the structural conformation of misfolded or unfolded proteins to induce these proteins to have normal biological functions. Deficiency in the chaperone protein may cause aging and Alzheimer's disease.

Meanwhile, microvesicles are substances with sizes of generally 0.03 to 1 μm, which are naturally released from the cell membranes in almost all types of cells, and are characterized to have a form of double phospholipid membrane, which is the structure of the cell membrane. These vesicles are basic tools of cells for metabolism, transport of metabolites, storage of enzymes, chemical reactions, and the like, and are known to serve to mediate cell-to-cell signaling by delivering mRNA, miRNA, proteins, and the like between cells.

Therefore, the vesicles that mediate the delivery of substances between cells have an excellent ability to deliver the contained substances to the cells. And thus, the demand for the vesicles which express and contain specific beneficial substances such as proteasome and CCT is gradually increasing, and there is a need for research thereabout.

### DISCLOSURE

### Technical Problem

Accordingly, the present inventors have studied microvesicles, microvesicles derived from a cell such as an exosome, and analogs thereof, and then completed a cell-derived vesicle containing an enriched protein such as proteasome and CCT as compared to an originated cell or an exosome naturally secreted from the originated cell.

Therefore, an object of the present disclosure is to provide a cell-derived vesicle rich in specific factors compared to a cell or a naturally derived exosome, and a method of preparing the same.

### Technical Solution

To achieve the object, the present disclosure provides a cell-derived vesicle rich in intracellular protein homeostasis regulators.

Further, the present disclosure provides a pharmaceutical composition comprising the cell-derived vesicle.

Further, the present disclosure provides a composition for drug delivery comprising the cell-derived vesicle.

Further, the present disclosure provides a method of preparing a cell-derived vesicle rich in intracellular protein homeostasis regulators comprising (1) preparing a cell-derived vesicle by transferring a sample containing cells into micropores.

### Advantageous Effects

According to the present disclosure, it is possible to easily maintain intracellular protein homeostasis by using the cell-derived vesicle rich in intracellular protein homeostasis regulators and the method of preparing the same to easily transfer the intracellular protein homeostasis regulators to, for example, a target such as a cell. Accordingly, the present disclosure can also be utilized for preventing or treating diseases caused by the homeostasis regulation failure of intracellular proteins or for preventing aging, and thus can be widely used in the drug industry.

### DESCRIPTION OF DRAWINGS

FIG. 1 is an exploded part view illustrating a self-made cell extruder for preparing a cell-derived vesicle of the present disclosure.

FIG. 2 is a diagram illustrating the number of proteins expressed in a U937 cell and a U937 cell-derived vesicle as a Venn diagram.

FIG. 3 is a diagram illustrating classification of proteins identified to be two times or more highly enriched in the U937 cell-derived vesicle compared to the U937 cell.

FIG. 4 is a diagram illustrating top 30 proteins among the proteins identified to be two times or more highly enriched in the U937 cell-derived vesicle compared to the U937 cell.

FIG. 5 is a diagram illustrating the number of proteins expressed in a U937 cell-derived naturally secreted exosome and a U937 cell-derived vesicle as a Venn diagram.

FIG. 6 is a diagram illustrating classification of proteins identified to be two times or more highly enriched in the U937 cell-derived vesicle compared to the U937 cell-derived naturally secreted exosome.

FIG. 7 is a diagram illustrating top 30 proteins among the proteins identified to be two times or more highly enriched in the U937 cell-derived vesicle compared to the U937 cell-derived naturally secreted exosome.

FIG. 8 is a diagram illustrating the number of proteins expressed in an adipose derived stem cell and an adipose derived stem cell-derived vesicle as a Venn diagram.

FIG. 9 is a diagram illustrating classification of proteins identified to be two times or more highly enriched in the adipose derived stem cell-derived vesicle compared to the adipose derived stem cell.

FIG. 10 is a diagram illustrating top 30 proteins among the proteins identified to be two times or more highly enriched in the adipose derived stem cell-derived vesicle compared to the adipose derived stem cell.

FIG. 11 is a diagram illustrating the number of proteins expressed in an adipose derived stem cell-derived naturally secreted exosome and an adipose derived stem cell-derived vesicle as a Venn diagram.

FIG. 12 is a diagram illustrating classification of proteins identified to be two times or more highly enriched in the adipose derived stem cell-derived vesicle compared to the adipose derived stem cell-derived naturally secreted exosome.

FIG. 13 is a diagram illustrating top 30 proteins among the proteins identified to be two times or more highly enriched in the adipose derived stem cell-derived vesicle compared to the adipose derived stem cell-derived naturally secreted exosome.

FIG. 14 is a diagram illustrating the number of proteins expressed in a human natural killer cell and a human natural killer cell-derived vesicle as a Venn diagram.

FIG. 15 is a diagram illustrating classification of proteins identified to be two times or more highly enriched in the human natural killer cell-derived vesicle compared to the human natural killer cell.

FIG. 16 is a diagram illustrating top 30 proteins among proteins identified to be two times or more highly enriched in the human natural killer cell-derived vesicle compared to the human natural killer cell.

FIG. 17 is a diagram illustrating expression rates of proteasome-related proteins in a cell-derived vesicle compared to an originated cell or a naturally secreted exosome.

FIG. 18 is a diagram illustrating classification of proteins two times or more highly enriched in a cell-derived vesicle compared to an originated cell and the proteins based on the originated cell as a Venn diagram.

FIG. 19 is a diagram illustrating classification of compositions of common proteins two times or more highly enriched in a cell-derived vesicle compared to an originated cell.

FIG. 20 is a diagram illustrating proteins classified into proteasome, CCT, dynectin, and exocyst among 64 proteins identified as commonly highly enriched in a cell-derived vesicle.

FIG. 21 is a diagram illustrating proteins two times or more highly enriched in a cell-derived vesicle compared to a naturally secreted exosome.

FIG. 22 is a diagram illustrating classification of 1153 proteins identified to be highly enriched in a cell-derived vesicle compared to a naturally secreted exosome regardless of an originated cell.

FIG. 23 is a diagram illustrating proteins classified into proteasome, CCT, dynectin, and exocyst among 1153 proteins.

### BEST MODE OF THE INVENTION

The present disclosure provides a cell-derived vesicle rich in intracellular protein homeostasis regulators.

The “intracellular protein homeostasis regulators” of the present disclosure refer to factors that maintain the structural conformation of a protein present in a cell. In order to complete its biological function, the protein needs to be folded into a precise three-dimensional conformation, which is regulated by the “intracellular protein homeostasis regulators”. Since a native conformation of the protein is encoded in its amino acid primary sequence, even a single mutation in an amino acid sequence may impair the ability of the protein to form its proper conformation. When the protein is not correctly folded, biological and clinical effects may be completely destroyed. The “intracellular protein homeostasis regulator” of the present disclosure means a factor that plays a role in decomposing, folding or assembling unnecessary, damaged, or misfolded proteins, preferably proteasome or chaperonin-containing T-complex polypeptide 1 (CCT).

In the present disclosure, the “cell-derived vesicle” may be rich in intracellular protein homeostasis regulators, preferably rich in proteasome or chaperonin-containing T-complex polypeptide 1 (CCT).

In the present disclosure, the “proteasome” is a protein complex that degrades unnecessary, damaged, or misfolded proteins. The proteasome functions to regulate the concentration of a specific protein in the cell. Proteins that require degradation are tagged by ubiquitin, and the proteasome recognizes a ubiquitin tag to degrade the tagged proteins, thereby maintaining the intracellular protein homeostasis. The proteasome is found in all eukaryotes and located in the nucleus and the cytoplasm. A proteasome degradation pathway is required for many cellular processes, including a cell cycle, regulation of gene expression and a response to oxidative stress. Proteasome deficiency causes several neurological diseases, including brain tumor, Parkinson's disease, and Alzheimer's disease. Neurodegenerative diseases are caused by misfolded proteins. Aggregates of the misfolded proteins may form insoluble aggregates, which may cause neurotoxicity. In addition, the impairment of the proteasome activity causes muscle disease. In addition, the genes encoding the “proteasome” of the present disclosure may include preferably at least one gene selected from the group consisting of PSMA1, PSMA2, PSMA3, PSMA4, PSMA5, PSMA6, PSMA7, PSMB1, PSMB10, PSMB2, PSMB3, PSMB4, PSMB5, PSMB6, PSMB7, PSMB8, PSMB9, PSMD11 and PSMD12, based on gene names denominated in the Gene card database (https://www.genecards.org/).

In the present disclosure, chaperonin-containing T-complex polypeptide 1 (CCT) is a protein constituting chaperone. The chaperone functions in the folding and assembly of protein conformations to maintain intracellular protein homeostasis. The chaperone prevents aggregation of proteins and adjusts the structural conformation of misfolded or unfolded proteins to induce these proteins to have normal biological functions. Deficiency in the chaperone protein may cause aging and Alzheimer's disease. In addition, the gene encoding “ ” of the present disclosure may include preferably at least one gene selected from the group consisting of CCT2, CCT3, CCT4, CCT5, CCT7 and CCT8, based on gene names denominated in the Gene card database (https://www.genecards.org/).

Accordingly, the cell-derived vesicle of the present disclosure may be a cell-derived vesicle rich in at least one selected from intracellular protein homeostasis regulators encoded by the genes shown in Table 1 below.

TABLE 1

| Gene symbol | Accession number |
|---|---|
| PSMA1 | P25786-2 |
| PSMA2 | P25787 |
| PSMA3 | P25788 |
| PSMA4 | P25789-2 |
| PSMA5 | P28066 |
| PSMA6 | P60900 |
| PSMA7 | O14818 |
| PSMB1 | P20618 |
| PSMB2 | P49721 |
| PSMB3 | P49720 |
| PSMB4 | P28070 |
| PSMB5 | P28074 |
| PSMB6 | P28072 |
| PSMB7 | Q99436 |
| PSMB8 | P28062 |
| PSMB9 | P28065 |
| PSMB10 | P40306 |
| PSMB11 | O00231 |
| PSMB12 | O00232 |
| CCT2 | P78371 |
| CCT3 | P49368 |
| CCT4 | P50991 |
| CCT5 | P48643 |
| CCT7 | Q99832 |
| CCT8 | P50990 |
| DCTN1 | Q14203 |
| DCTN2 | Q13561-2 |
| EXOC3L4 | Q17RC7 |
| EXOC7 | Q9UPT5 |

In addition, in the present disclosure, the intracellular protein homeostasis regulators may be encoded by at least one gene selected from the group consisting of PSMA1, PSMA2, PSMA3, PSMA4, PSMA5, PSMA6, PSMA7, PSMB1, PSMB10. PSMB2, PSMB3, PSMB4, PSMB5, PSMB6, PSMB7, PSMB8, PSMB9, PSMD11 and PSMD12.

In addition, the intracellular protein homeostasis regulators may be encoded by at least one gene selected from the group consisting of CCT2, CCT3, CCT4, CCT5, CCT7 and CCT8.

In the present disclosure, the “cell-derived vesicle” may be rich, preferably two times or more rich in intracellular protein homeostasis regulators, compared to an originated cell or an exosome naturally secreted from the originated cell.

In the present disclosure, the “cell-derived vesicle” is a vesicle artificially prepared in a cell and has the form of a double phospholipid membrane, which is the structure of a cell membrane. The cell-derived vesicle of the present disclosure may be rich in intracellular protein homeostasis regulators to maintain the homeostasis of intracellular proteins of a target cell, and may exhibit an effect of treating a disease of an individual based on the effect of regulating intracellular protein homeostasis.

In order to prepare the cell-derived vesicle of the present disclosure, a nucleated cell or a transformed cell thereof may be used, but any cell capable of preparing a vesicle may be used without limitation, preferably a stem cell, an undifferentiated cell, an immune cell, a somatic cell, a dedifferentiated stem cell or a germ cell. In the present disclosure, as a specific example embodiment, a U937 cell, an adipose derived stem cell (ADSC) and a human natural killer cell (human NK cell) were used.

In the present disclosure, the exosome naturally secreted from the originated cell refers to an exosome secreted into a medium by cells cultured by a conventional cell culture method, unlike the cell-derived vesicle of the present disclosure prepared by transferring the cells present in the sample to micropores, and refers to an exosome obtained through centrifugation of the medium.

The vesicle of the present disclosure may be prepared by using a method selected from the group consists of extruding a suspension containing cells, sonicating, lysing cells, homogenizing, freeze-thawing, electroporation, chemical treatment, mechanical decomposition, and treatment with a physical stimulus applied externally to the cells, but is not limited thereto. In the present disclosure, the vesicle was prepared by using a cell extruder of a micropore extrusion method, which sequentially transfers and extrudes the suspension containing the cells from a filter with a large pore size to a filter with a small pore size by applying pressure.

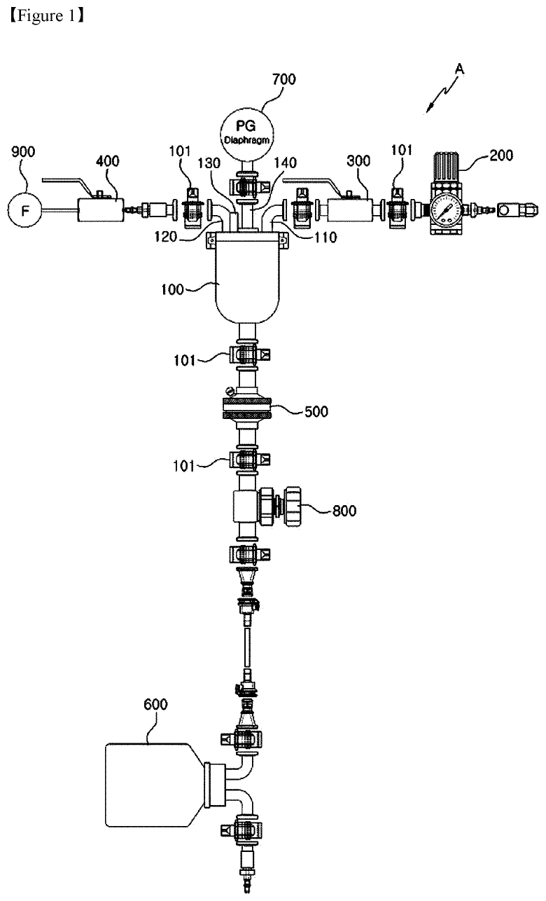

An exploded part diagram illustrating a cell extruder used for preparing the vesicle is illustrated in FIG. 1. As illustrated in FIG. 1, the cell extruder is configured by a dispensing pressure vessel 100, a regulator 200 for maintaining a static pressure after adjusting gas injected into the pressure vessel 100 to a set pressure, an input valve 300 for opening and closing the injection of gas into the pressure vessel 100, an exhaust valve 400 for removing the internal pressure of the pressure vessel, a filter holder 500 having a membrane filter soaked with a reagent so that a sample in the pressure vessel 100 is pumped and passed by gas and the sample is crushed to a size of microvesicles, and a collection vessel 600 in which the extruded sample is stored.

Further, the present disclosure provides a pharmaceutical composition comprising the cell-derived vesicle.

The pharmaceutical composition of the present disclosure is based on the fact that the cell-derived vesicle contains high concentrations of proteasome and CCT, and may be utilized for “a use for the prevention or treatment of disease caused by an intracellular protein homeostasis regulation failure” or “a use for preventing aging”. The “disease caused by the intracellular protein homeostasis regulation failure” may be preferably brain tumor, amyotrophic lateral sclerosis, Alzheimer's disease, Parkinson's disease, prion disease, polyglutamine ectasia, spinal cerebellar ataxia, spinal cord and medulla oblongata, spongiform encephalopathy, tauopathy, Huntington's disease, or dystonia.

Accordingly, the present disclosure may provide a method for preventing or treating disease caused by an intracellular protein homeostasis regulation failure comprising treating the cell-derived vesicle to a subject.

In addition, the present disclosure may provide a method for preventing aging comprising treating the cell-derived vesicle to a subject.

The pharmaceutical composition of the present disclosure may use a pharmaceutically acceptable carrier, that is, saline, sterile water, a Ringer's solution, buffered saline, cyclodextrin, a dextrose solution, a maltodextrin solution, glycerol, ethanol, liposome, and a combination of one or more of these ingredients in addition to the cell-derived vesicle, and may further include other conventional additives such as an antioxidant and a buffer as needed. In addition, the pharmaceutical composition may be prepared in injectable formulations such as an aqueous solution, a suspension, and an emulsion, pills, capsules, granules, or tablets by further adding a diluent, a dispersant, a surfactant, a binder, and/or a lubricant. Furthermore, the pharmaceutical composition may be prepared preferably according to each ingredient by using a suitable method of the art, or a method disclosed in Remington's Pharmaceutical Science (Mack Publishing Company, Easton PA). The pharmaceutical composition of the present disclosure is not particularly limited in formulation, but is preferably formulated as an injection or inhalant.

An administration method of the pharmaceutical composition of the present disclosure is not particularly limited, but the pharmaceutical composition may be administered parenterally or orally, such as intravenous, subcutaneous, intraperitoneal, inhalation or topical application, depending on a desired method. The dose thereof varies in the range thereof according to the weight, age, sex, health condition, diet, administration time, administration method, and excretion rate of a patient, the severity of disease, etc. A daily dose means an amount of a therapeutic agent of the present disclosure sufficient to treat a disease state alleviated by administration to a subject in need of the treatment. The effective amount of the therapeutic agent depends on a particular compound, a disease state and its severity, and a subject in need of treatment, which may be generally determined by those skilled in the art. As a non-limiting example, the dose for the human body of the composition according to the present disclosure may vary depending on the patient's age, weight, sex, dosage form, health condition, and disease level, and may be dividedly administered once to several times a day at regular time intervals, based on an adult patient having a body weight of 70 kg.

In the present disclosure, the “subject” refers to a subject in need of the prevention or treatment of disease caused by the intracellular protein homeostasis regulation failure” or “preventing aging”, and more particularly, refers to mammals such as human or non-human primates, mice, rats, dogs, cats, horses and cattle.

Further, the present disclosure provides a composition for drug delivery comprising the cell-derived vesicle.

A drug delivery carrier of the present disclosure includes the cell-derived vesicle described above, and the contents duplicated with the description of the cell-derived vesicle will be omitted. The drug delivery carrier according to the present disclosure includes the above-described cell-derived vesicle to have excellent storage stability and low cytotoxicity. In particular, the drug delivery carrier including the cell-derived vesicle of the present disclosure contains proteasome or CCT in a higher content than the originated cell or the exosome naturally secreted from the originated cell to effectively deliver the proteasome or CCT, thereby achieving an excellent therapeutic effect according to the effective delivery of the proteasome or CCT.

The drug that may be carried in the drug delivery carrier of the present disclosure may be included without limitation, as long as the drug is a substance capable of expressing its desired effect by being delivered into a cell, but may be preferably any one or more selected from the group consisting of an antifungal agent, an antibacterial agent, an antimicrobial agent, an antioxidant, a cooling agent, a soothing agent, a wound healing agent, an anti-inflammatory agent, an anti-aging agent, an anti-wrinkle agent, a skin whitening agent, an anticancer agent, an angiogenesis inhibitor, peptides, proteins, toxins, nucleic acids, beads, and microparticle and nanoparticle proteins and compounds.

Further, the present disclosure provides a method of preparing a cell-derived vesicle rich in intracellular protein homeostasis regulators comprising (1) preparing a cell-derived vesicle by transferring a sample containing cells into micropores.

According to the preparing method, it is possible to prepare a cell-derived vesicle rich in at least one selected from intracellular protein homeostasis regulators encoded by genes shown in Table 1. Particularly, it is possible to prepare a cell-derived vesicle two times or more rich in intracellular protein homeostasis regulators than the originated cell or the exosome naturally secreted from the originated cell.

The intracellular protein homeostasis regulators may be encoded by at least one gene selected from the group consisting of PSMA1, PSMA2, PSMA3, PSMA4, PSMA5, PSMA6, PSMA7, PSMB1, PSMB10, PSMB2, PSMB3, PSMB4, PSMB5, PSMB6, PSMB7, PSMB8, PSMB9, PSMD11 and PSMD12, or encoded by at least one gene selected from the group consisting of CCT2, CCT3, CCT4, CCT5, CCT7 and CCT8.

The originated cell may be a non-nucleated cell or a nucleated cell, preferably a stem cell, an undifferentiated cell, an immune cell, a somatic cell, a dedifferentiated stem cell or a germ cell.

The preparing method may further include selecting a cell-derived vesicle rich in the intracellular protein homeostasis regulators from the cell-derived vesicles prepared in step (1).

The “sample containing the cells” of the present disclosure may be a sample containing a non-nucleated cell, a nucleated cell, or a transformed cell thereof, and may be a sample containing cells capable of preparing the vesicle without limitation.

In step (1) of the present disclosure, the “preparing of the cell-derived vesicle by transferring the sample into the micropores” may be a step of transferring the sample by applying a pressure to a filter having a filterable membrane structure with a pore size of 1 to 10 μm, and preferably a step of transferring the sample sequentially from a filter having a large pore size to a filter having a small pore size. That is, the transferring into the micropores may be performed by pressure.

The selecting step of the present disclosure may be selecting the cell-derived vesicle particularly rich in intracellular protein homeostasis regulators among the cell-derived vesicles in the sample subjected to the transferring step of step (1), preferably selecting the cell-derived vesicle particularly rich in proteasome and/or CCT. In order to perform the selecting step of the present disclosure, a conventional proteomics analysis method in the art, such as mass spectrometry (MS), liquid chromatography-mass spectrometry (LC/MS), or nuclear magnetic resonance (NMR) analysis method may be used, and in an example embodiment of the present disclosure, the cell-derived vesicle was measured by an LC/MS method.

According to the preparing method of the present disclosure, it is possible to prepare a cell-derived vesicle rich in protein homeostasis regulators that may be used for preventing or treating disease caused by the intracellular protein homeostasis regulation failure or for preventing aging.

Duplicated contents are omitted in consideration of the complexity of the present specification, and terms not defined otherwise in the present specification have the meanings commonly used in the art to which the present disclosure pertains.

### MODES OF THE INVENTION

### Example Embodiment 1. Preparation of Samples

Samples were prepared with three types of a cell, a naturally secreted exosome, and a cell-derived vesicle (CDV), and originated cells of each sample were a U937 cell, an adipose derived stem cell (Lonza), and a human natural killer cell isolated from a healthy volunteer blood, respectively.

For preparation of a cell sample, cells were cultured in a nutrient medium for at least 7 days and proliferated so that the total number of cells was 1×10⁸ cells or more. One day before cell harvest, a DMEM (Gibco, #11995-073) nutrient medium added with 10% FBS and 1% penicillin-streptomycin was replaced with a DMEM (Gibco, #11995-073) pure medium, and after 24 hours, the pure medium was harvested to obtain a naturally secreted exosome secreted from the cells. Some of the cells were subdivided for proteomic samples of the cells themselves after harvest, and most of the cells were diluted in PBS at a concentration of 1×10⁶ cells/mL, and then introduced to an extrusion process to produce cell-derived vesicles. The naturally secreted exosome was isolated and obtained through continuous centrifugation at 300 g, 2000 g, 10000 g, and 120000 g. The extrusion process for producing cell-derived vesicles was performed with a gas-type cell extruder, and purified by tangential flow filtration (TFF) to prepare cell-derived vesicles. An exploded view of the gas-type cell extruder was illustrated in FIG. 1.

### Example Embodiment 2. Searching for Enriched Substances Contained in Cell-Derived Vesicles

### 2.1 Identification of Expression Substances in Sample

With respect to the cell, naturally secreted exosome, and cell-derived vesicle samples prepared in the same manner as in Example embodiment 1, proteomics analysis for identifying substances expressed and contained in each sample was performed. Specifically, sampling of each sample was performed. Sampling was performed through western blot, and each sample was treated with an RIPA buffer and an SDS sample buffer. The samples treated on a polyacrylamide gel were electrophoresed, and the gel was cut to obtain protein ingredients of each sample. Thereafter, the protein ingredients of each sample were analyzed through LC-MS analysis. However, the human NK cells had a very small secretion of the naturally secreted exosome, and the human NK cell-derived naturally secreted exosome was not analyzed.

### 2.2. Analysis of Proteins Enriched in U937 Cell-Derived Vesicle

### 2.2.1. Analysis of Proteins Enriched in U937 Cell-Derived Vesicle Compared to U937 Cell

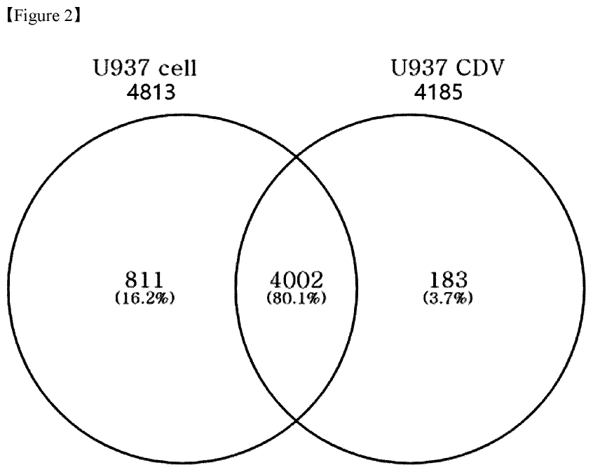

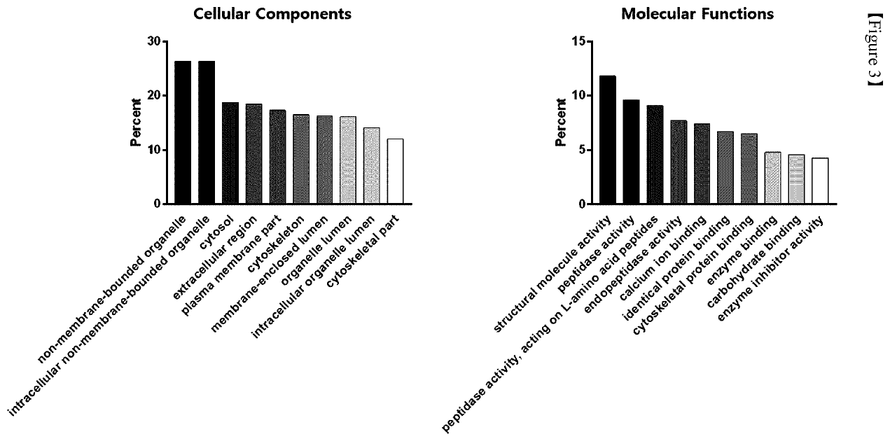

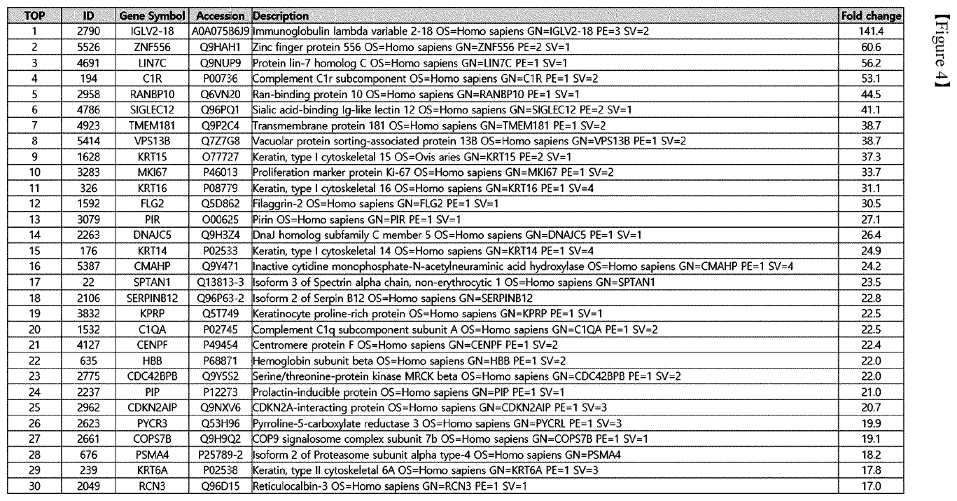

Based on the results of the analysis in Example embodiment 2.1, the number of proteins expressed in a U937 cell and a U937 cell-derived vesicle was illustrated in FIG. 2 as a Venn diagram. The classification of proteins identified to be two times or more highly enriched in the U937 cell-derived vesicle compared to the U937 cell was illustrated in FIG. 3. Top 30 proteins among the proteins identified to be two times or more highly enriched in the U937 cell-derived vesicle compared to the U937 cell was illustrated in FIG. 4.

### 2.2.2. Analysis of Proteins Enriched in U937 Cell-Derived Vesicle Compared to U937 Cell-Derived Naturally Secreted Exosome

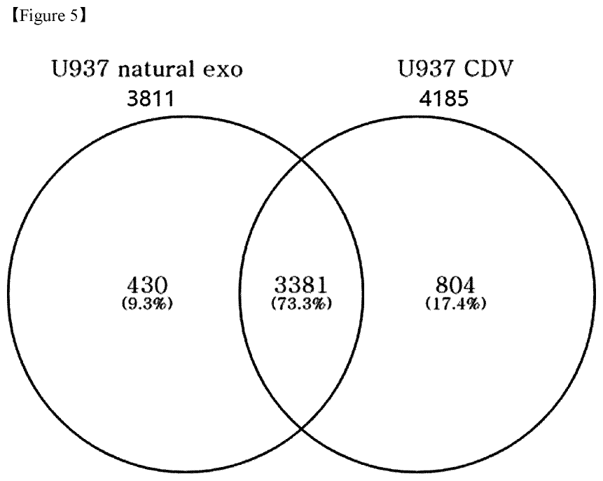

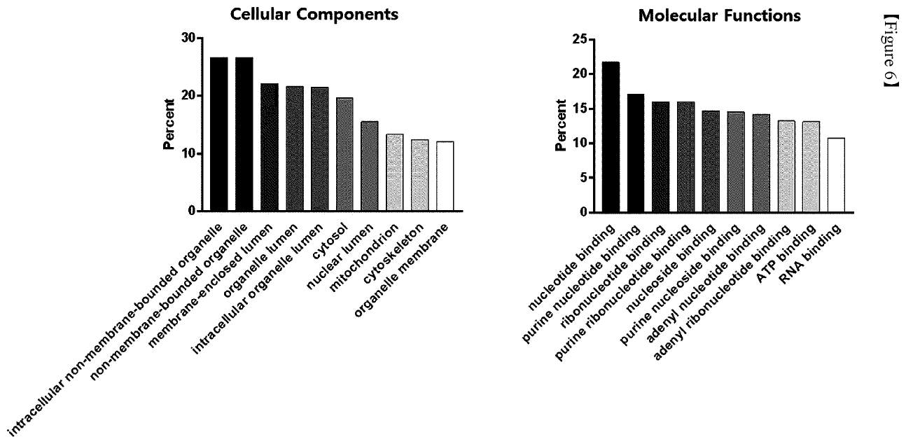

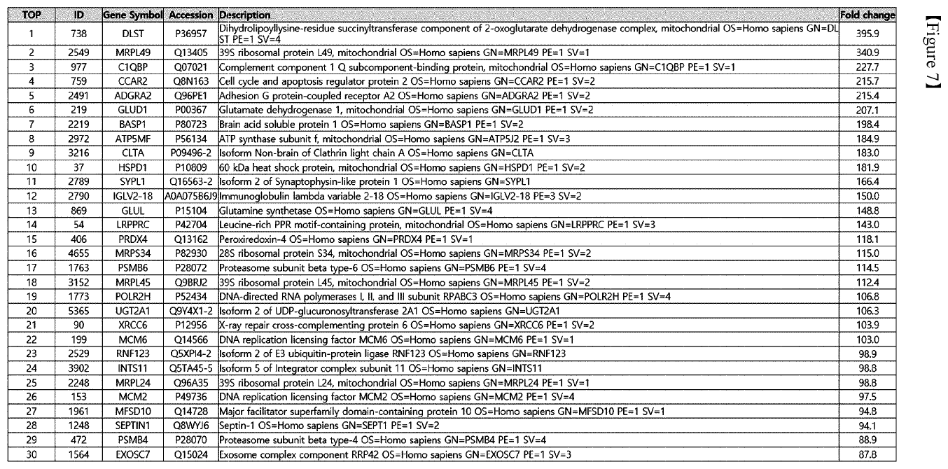

Based on the results of the analysis in Example embodiment 2.1, the number of proteins expressed in a U937 cell-derived naturally secreted exosome and a U937 cell-derived vesicle was illustrated in FIG. 5 as a Venn diagram. The classification of proteins identified to be two times or more highly enriched in the U937 cell-derived vesicle compared to the U937 cell-derived naturally secreted exosome was illustrated in FIG. 6. Top 30 proteins among the proteins identified to be two times or more highly enriched in the U937 cell-derived vesicle compared to the U937 cell-derived naturally secreted exosome were illustrated in FIG. 7.

### 2.3. Analysis of Proteins Enriched in Adipose Derived Stem Cell-Derived Vesicle

### 2.3.1. Analysis of Proteins Enriched in Adipose Derived Stem Cell-Derived Vesicle Compared to Adipose Derived Stem Cell

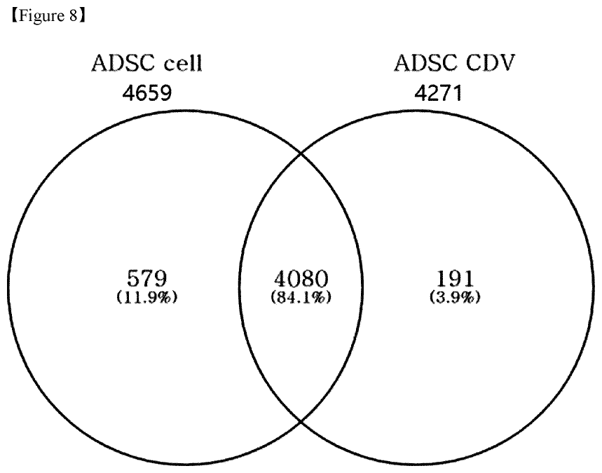

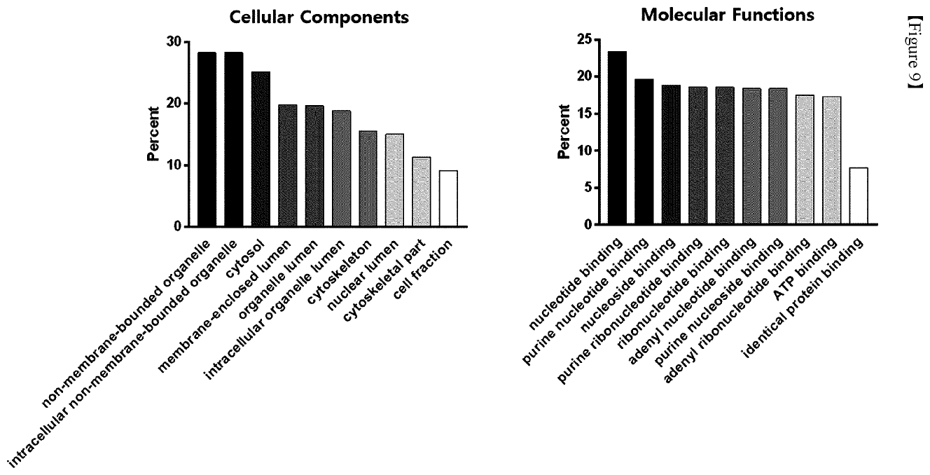

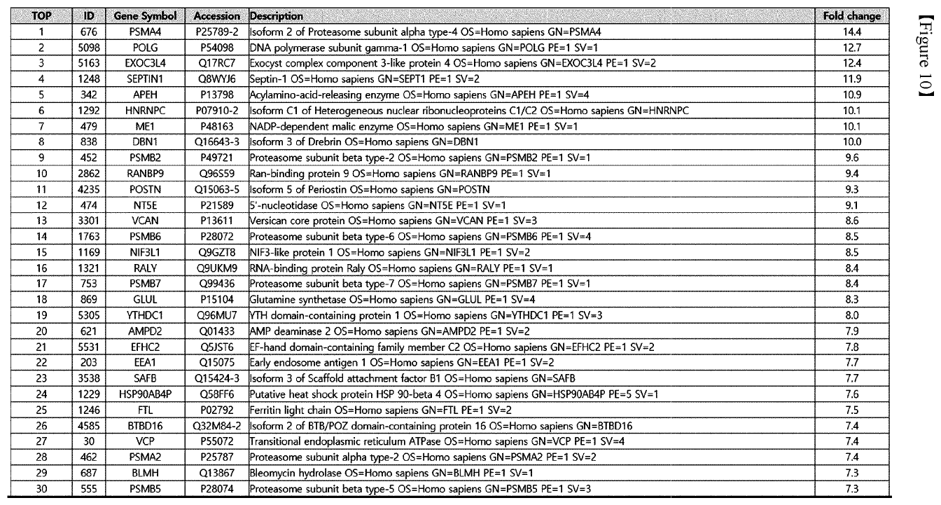

Based on the results of the analysis in Example embodiment 2.1, the number of proteins expressed in an adipose derived stem cell and an adipose derived stem cell-derived vesicle was illustrated in FIG. 8 as a Venn diagram. The classification of proteins identified to be two times or more highly enriched in the adipose derived stem cell-derived vesicle compared to the adipose derived stem cell was illustrated in FIG. 9. Top 30 proteins among the proteins identified to be two times or more highly enriched in the adipose derived stem cell-derived vesicle compared to the adipose derived stem cell were illustrated in FIG. 10.

### 2.3.2. Analysis of Proteins Enriched in Adipose Derived Stem Cell-Derived Vesicle Compared to Adipose Derived Stem Cell-Derived Naturally Secreted Exosome

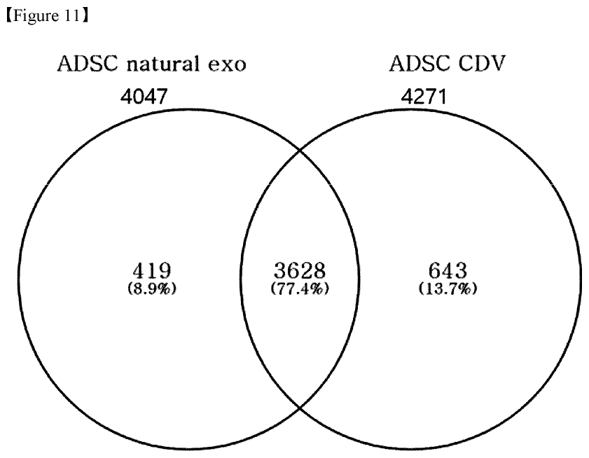

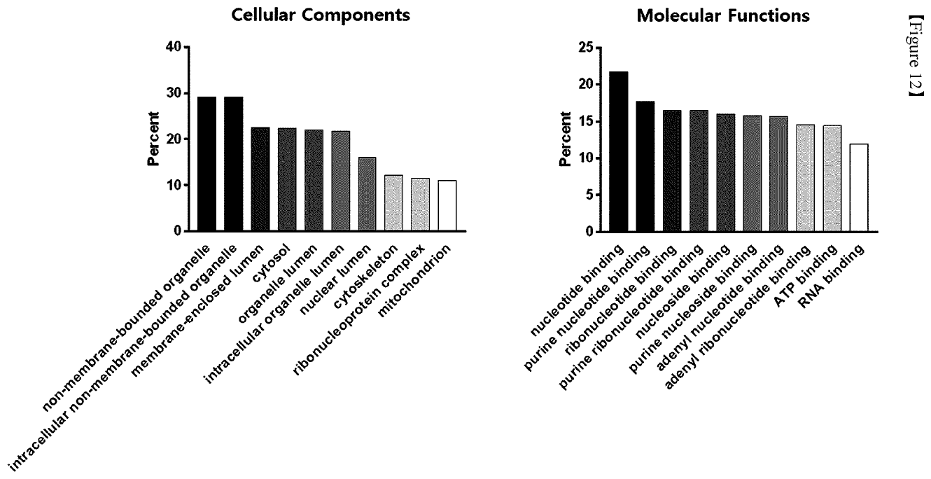

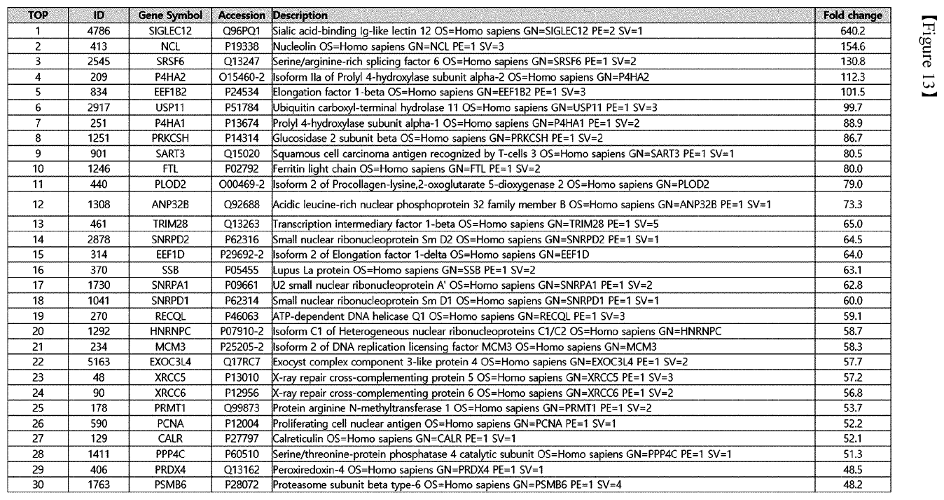

Based on the results of the analysis in Example embodiment 2.1, the number of proteins expressed in an adipose derived stem cell-derived naturally secreted exosome and an adipose derived stem cell-derived vesicle was illustrated in FIG. 11 as a Venn diagram. The classification of proteins identified to be two times or more highly enriched in the adipose derived stem cell-derived vesicle compared to the adipose derived stem cell-derived naturally secreted exosome was illustrated in FIG. 12. Top 30 proteins among the proteins identified to be two times or more highly enriched in the adipose derived stem cell-derived vesicle compared to the adipose derived stem cell-derived naturally secreted exosome were illustrated in FIG. 13.

### 2.4. Analysis of Proteins Enriched in Human NK Cell-Derived Vesicle

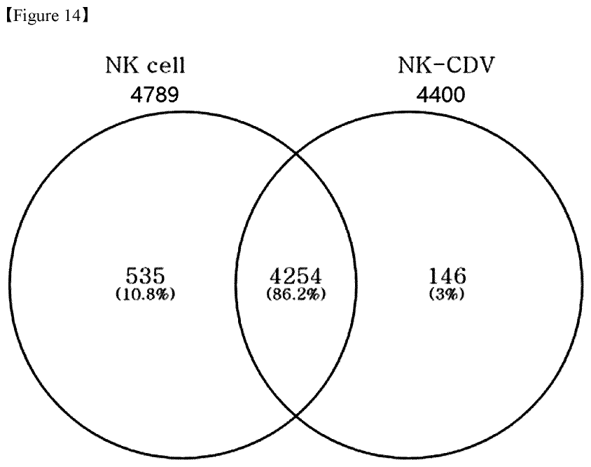

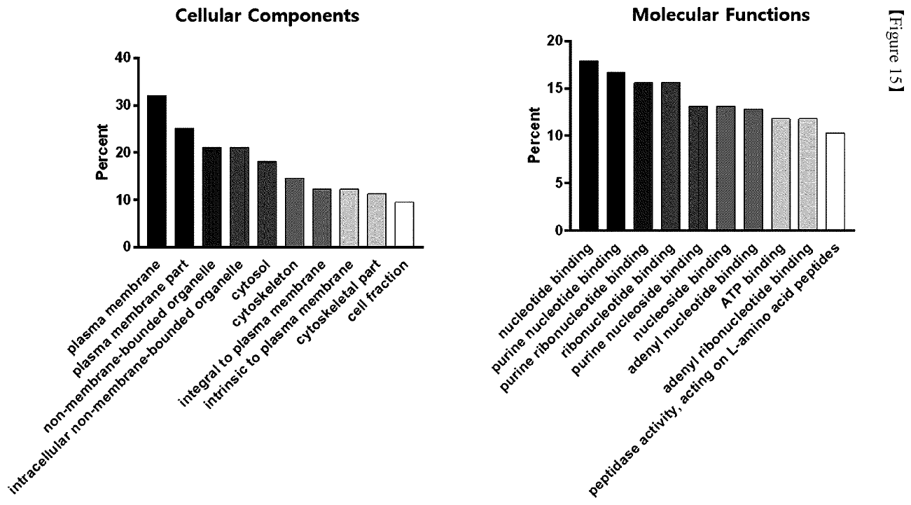

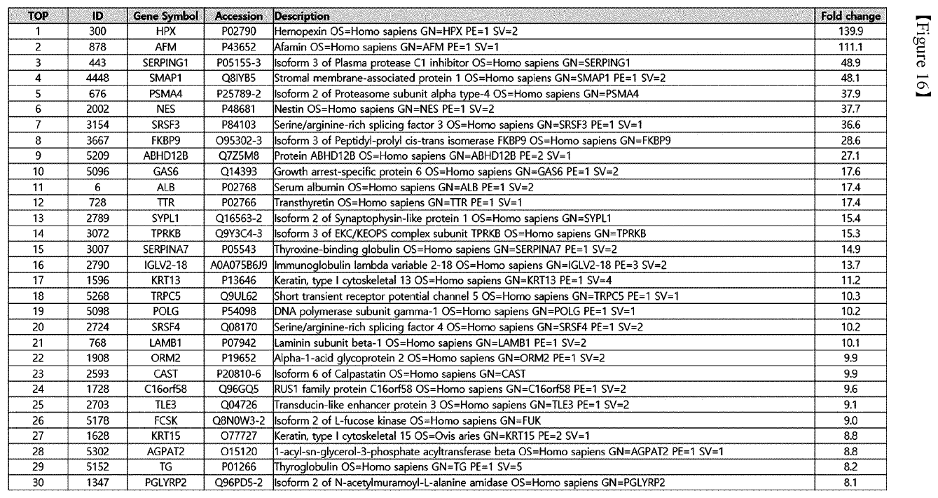

Based on the results of the analysis in Example embodiment 2.1, the number of proteins expressed in a human NK cell and a human NK cell-derived vesicle was illustrated in FIG. 14 as a Venn diagram. The classification of proteins identified to be two times or more highly enriched in the human NK cell-derived vesicle compared to the human NK cell was illustrated in FIG. 15. Top 30 proteins among proteins identified to be two times or more highly enriched in the human NK cell-derived vesicle compared to the human NK cell were illustrated in FIG. 16.

From the results confirmed in Example embodiments 2.2 to 2.4, it was confirmed that the U937 cell-derived vesicle and the adipose derived stem cell-derived vesicle were enriched in a non-membrane bounded organelle protein compared to the cell or the naturally secreted exosome, and the human NK cell-derived vesicle was enriched in a plasma membrane protein compared to the cell or the naturally secreted exosome.

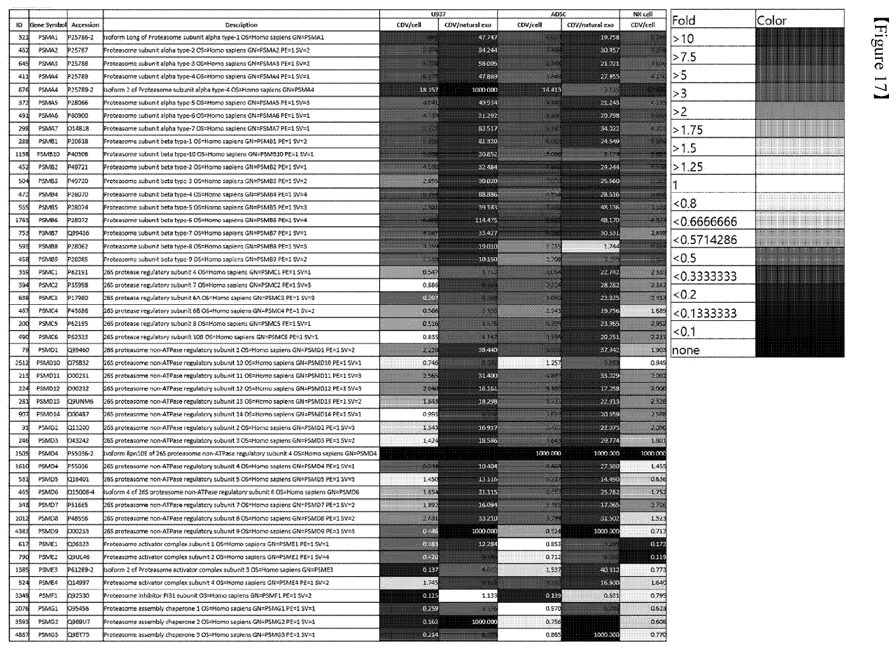

In addition, for proteasome-related proteins, the expression rate of the cell-derived vesicle compared to the cell or the naturally secreted exosome was illustrated in FIG. 17.

As illustrated in FIG. 17, in terms of the expression rate of the proteasome-related proteins, it was confirmed that the cell-derived vesicle was more similar to the cell than the naturally secreted exosome.

### Example Embodiment 3. Analysis of Proteins Commonly Enriched in Cell-Derived Vesicles

### 3.1. Analysis of Proteins Commonly Enriched in Cell-Derived Vesicle Compared to Originated Cell

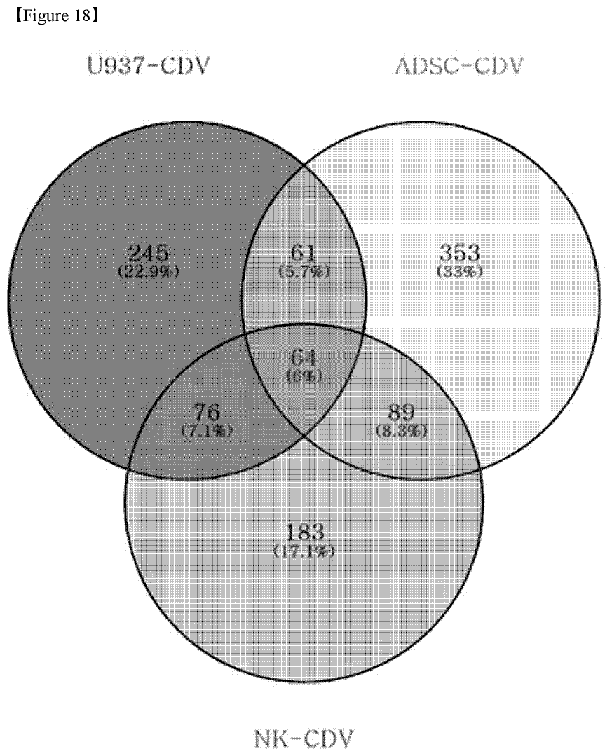

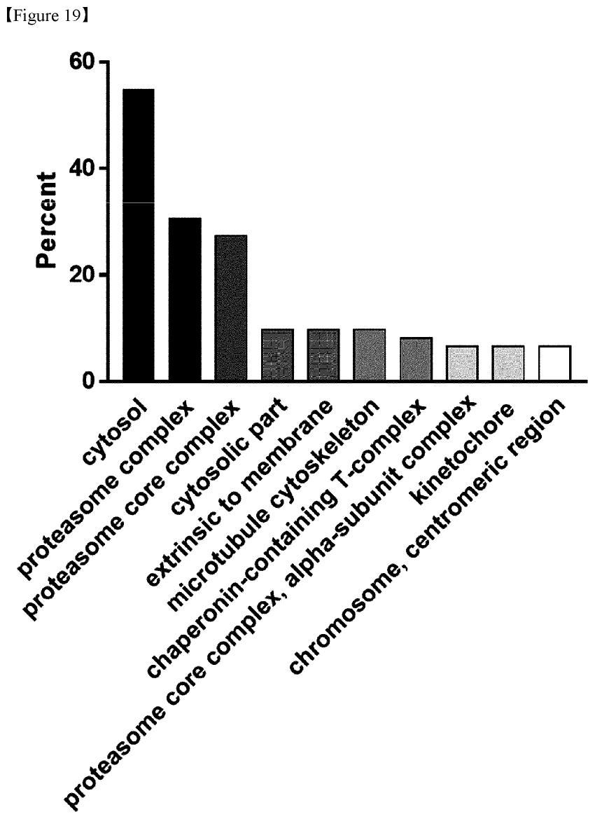

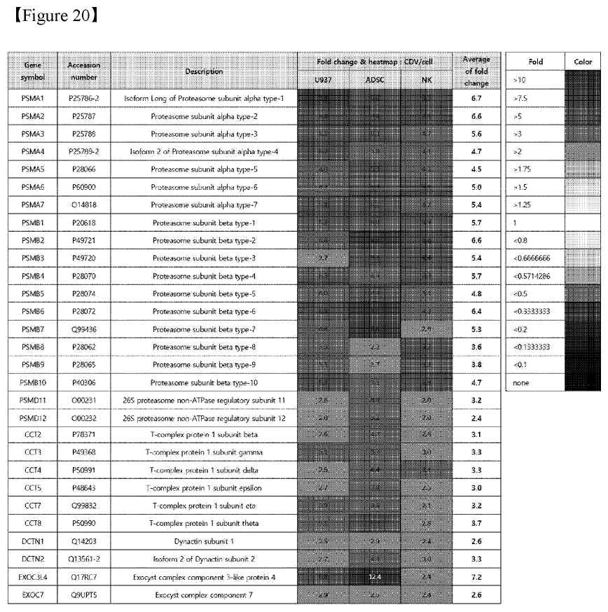

Based on the results analyzed in Example embodiment 2.1, proteins two times or more highly enriched in a cell-derived vesicle compared to an originated cell were classified and the number of proteins was illustrated in FIG. 18 as a Venn diagram based on the originated cell. The classification of 64 proteins identified to be commonly highly enriched in a cell-derived vesicle was illustrated in FIG. 19, and among the 64 proteins, proteins classified into proteasome, chaperonin-containing T-complex polypeptide 1 (CCT), dynectin, and exocyst were illustrated in FIG. 20.

As illustrated in FIG. 18, it was confirmed that the U937 cell-derived vesicle highly contained 446 proteins compared to the U937 cell. In addition, it was confirmed that the adipose derived stem cell-derived vesicle highly contained 567 proteins compared to the adipose derived stem cell. In addition, it was confirmed that the human NK cell-derived vesicle highly contained 412 proteins compared to the human NK cell. In addition, it was confirmed that 64 proteins were commonly highly contained in the cell-derived vesicle, regardless of the originated cell.

As illustrated in FIGS. 19 and 20, it was confirmed that among 64 proteins identified to be commonly highly contained in the cell-derived vesicle regardless of the originated cell, the proteins classified into proteasome, chaperonin-containing T-complex polypeptide 1 (CCT), dynectin, and exocyst were particularly highly enriched, and the number of concentrations reached 2 to 9.6 times higher than that of the cell.

### 3.2. Analysis of Proteins Commonly Enriched in Cell-Derived Vesicle Compared to Naturally Secreted Exosome

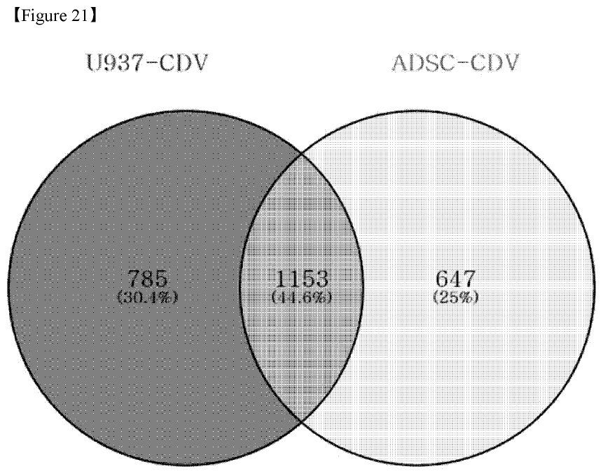

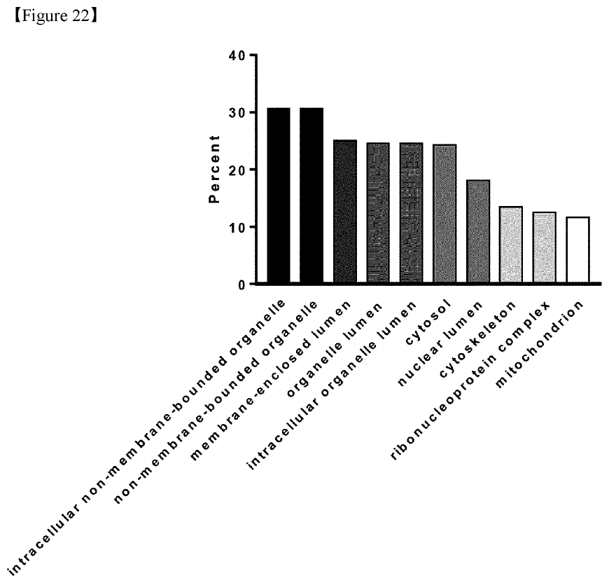

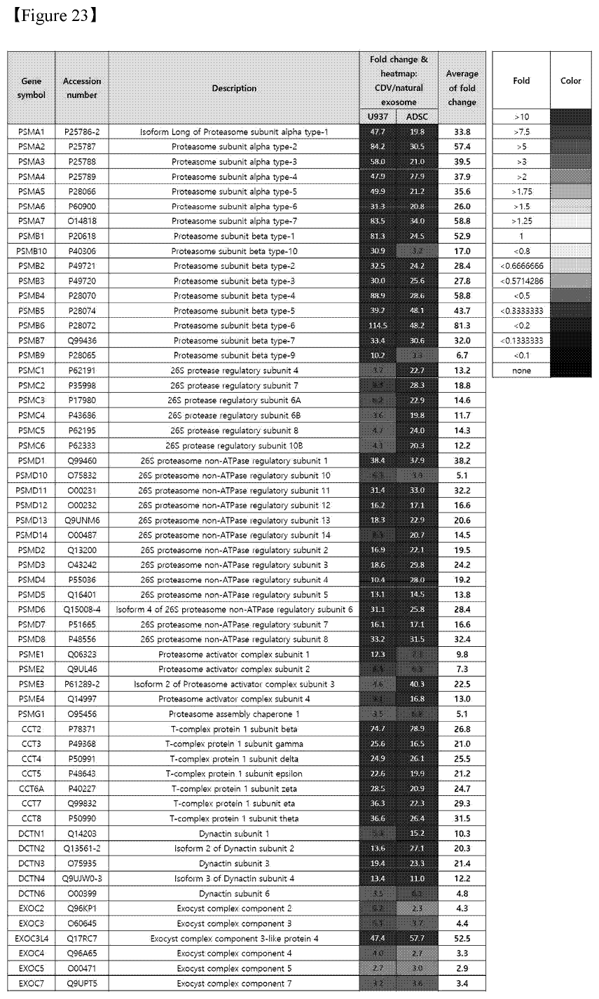

Based on the results analyzed in Example embodiment 2.1, proteins two times or more highly enriched in a cell-derived vesicle compared to a naturally secreted exosome were identified and the results thereof were illustrated in FIG. 21. The classification of 1153 proteins identified to be highly enriched in a cell-derived vesicle compared to a naturally secreted exosome regardless of an originated cell was illustrated in FIG. 22. Among the 1153 proteins, proteins classified into proteasome, chaperonin-containing T-complex polypeptide 1 (CCT), dynectin, and exocyst were illustrated in FIG. 23.

As illustrated in FIG. 21, it was confirmed that the U937 cell-derived vesicle highly contained 1938 proteins compared to the U937 cell-derived naturally secreted exosome. In addition, it was confirmed that the adipose derived stem cell-derived vesicle highly contained 1800 proteins compared to the adipose derived stem cell-derived naturally secreted exosome. In addition, it was confirmed that 1153 proteins were commonly highly contained in the cell-derived vesicle, regardless of the originated cell.

As illustrated in FIGS. 22 and 23, it was confirmed that among 1153 proteins identified to be commonly highly contained in the cell-derived vesicle compared to the naturally secreted exosome regardless of the originated cell, the proteins classified into proteasome, chaperonin-containing T-complex polypeptide 1 (CCT), dynectin, and exocyst were particularly highly enriched, and the number of concentration reached 2 to 80 times higher than that of the cell.

### Preparation Example of Drug

The pharmaceutical composition according to the present disclosure may be formulated in various forms depending on the purposes. The following examples illustrate several formulation methods containing the cell-derived vesicle according to the present disclosure as an active ingredient, but the present disclosure is not limited thereto.

### <1-1> Preparation of Powders

Cell-derived vesicles of the present disclosure 2 g
Lactose 1 g

The above ingredients were mixed and filled in an airtight bag to prepare powders.

### <1-2> Preparation of Tablets

Cell-derived vesicles of the present disclosure 100 mg
Corn starch 100 mg
Lactose 100 mg
Magnesium stearate 2 mg

The above ingredients were mixed and then tableted according to a general tablet preparing method to prepare tablets.

### <1-3> Preparation of Capsules

Cell-derived vesicles of the present disclosure 100 mg
Corn starch 100 mg
Lactose 100 mg
Magnesium stearate 2 mg

The above ingredients were mixed and then filled in gelatin capsules according to a general capsule preparing method to prepare capsules.

### <1-4> Preparation of Pills

Cell-derived vesicles of the present disclosure 1 g
Lactose 1.5 g
Glycerin 1 g
Xylitol 0.5 g

The above ingredients were mixed and then prepared to be 4 g per 1 pill according to a general method.

### <1-5> Preparation of Granules

Cell-derived vesicles of the present disclosure 50 mg
Soybean extract 50 mg
Glucose 200 mg
Starch 600 mg

The above ingredients were mixed and then added with 100 mg of 30% ethanol and dried at 60° C. to form granules and fill the granules in bags.

As described above, specific parts of the present disclosure have been described in detail, and it will be apparent to those skilled in the art that these specific techniques are merely preferred example embodiments, and the scope of the present disclosure is not limited thereto. Therefore, the substantial scope of the present disclosure will be defined by the appended claims and their equivalents.

1. A cell-derived vesicle rich in intracellular protein homeostasis regulators.

2. The cell-derived vesicle of claim 1, wherein the intracellular protein homeostasis regulators are at least one selected from intracellular protein homeostasis regulators encoded by genes shown in Table below.
TABLE 1

| Gene symbol | Accession number |
|---|---|
| PSMA1 | P25786-2 |
| PSMA2 | P25787 |
| PSMA3 | P25788 |
| PSMA4 | P25789-2 |
| PSMA5 | P28066 |
| PSMA6 | P60900 |
| PSMA7 | O14818 |
| PSMB1 | P20618 |
| PSMB2 | P49721 |
| PSMB3 | P49720 |
| PSMB4 | P28070 |
| PSMB5 | P28074 |
| PSMB6 | P28072 |
| PSMB7 | Q99436 |
| PSMB8 | P28062 |
| PSMB9 | P28065 |
| PSMB10 | P40306 |
| PSMB11 | O00231 |
| PSMB12 | O00232 |
| CCT2 | P78371 |
| CCT3 | P49368 |
| CCT4 | P50991 |
| CCT5 | P48643 |
| CCT7 | Q99832 |
| CCT8 | P50990 |
| DCTN1 | Q14203 |
| DCTN2 | Q13561-2 |
| EXOC3L4 | Q17RC7 |
| EXOC7 | Q9UPT5. |

3. The cell-derived vesicle of claim 1, wherein the intracellular protein homeostasis regulators are enriched two times or more compared to an originated cell.

4. The cell-derived vesicle of claim 1, wherein the intracellular protein homeostasis regulators are enriched two times or more compared to an exosome naturally secreted from the originated cell.

5. The cell-derived vesicle of claim 2, wherein the intracellular protein homeostasis regulators are encoded by at least one gene selected from the group consisting of PSMA1, PSMA2, PSMA3, PSMA4, PSMA5, PSMA6, PSMA7, PSMB1, PSMB10, PSMB2, PSMB3, PSMB4, PSMB5, PSMB6, PSMB7, PSMB8, PSMB9, PSMD11 and PSMD12.

6. The cell-derived vesicle of claim 2, wherein the intracellular protein homeostasis regulators are encoded by at least one gene selected from the group consisting of CCT2, CCT3, CCT4, CCT5, CCT7 and CCT8.

7. The cell-derived vesicle of claim 1, wherein the vehicle is derived from a non-nucleated cell or a nucleated cell.

8. The cell-derived vesicle of claim 7, wherein the non-nucleated cell or the nucleated cell is a stem cell, an undifferentiated cell, an immune cell, a somatic cell, a dedifferentiated stem cell or a germ cell.

9. The cell-derived vesicle of claim 1, wherein the vehicle is prepared by transferring cells presented in a sample into micropores.

10. A method for treating disease caused by an intracellular protein homeostasis regulation failure comprising the cell-derived vesicle of claim 1.

11. The method of claim 10, wherein the disease caused by an intracellular protein homeostasis regulation failure is any one or more diseases selected from the group consisting of brain tumor, amyotrophic lateral sclerosis, Alzheimer's disease, Parkinson's disease, prion disease, polyglutamine ectasia, spinal cerebellar ataxia, spinal cord and medulla oblongata, spongiform encephalopathy, tauopathy, Huntington's disease, and dystonia.

12. The cell-derived vesicle of claim 1, wherein the vesicle is formulated as a drug delivery carrier.

13. A method of preparing a cell-derived vesicle rich in intracellular protein homeostasis regulators comprising (1) preparing a cell-derived vesicle by transferring a sample containing cells into micropores.

14. The method of preparing the cell-derived vesicle of claim 13, wherein the intracellular protein homeostasis regulators comprise at least one selected from intracellular protein homeostasis regulators encoded by genes shown in Table below.
TABLE 1

| Gene symbol | Accession number |
|---|---|
| PSMA1 | P25786-2 |
| PSMA2 | P25787 |
| PSMA3 | P25788 |
| PSMA4 | P25789-2 |
| PSMA5 | P28066 |
| PSMA6 | P60900 |
| PSMA7 | O14818 |
| PSMB1 | P20618 |
| PSMB2 | P49721 |
| PSMB3 | P49720 |
| PSMB4 | P28070 |
| PSMB5 | P28074 |
| PSMB6 | P28072 |
| PSMB7 | Q99436 |
| PSMB8 | P28062 |
| PSMB9 | P28065 |
| PSMB10 | P40306 |
| PSMB11 | O00231 |
| PSMB12 | O00232 |
| CCT2 | P78371 |
| CCT3 | P49368 |
| CCT4 | P50991 |
| CCT5 | P48643 |
| CCT7 | Q99832 |
| CCT8 | P50990 |
| DCTN1 | Q14203 |
| DCTN2 | Q13561-2 |
| EXOC3L4 | Q17RC7 |
| EXOC7 | Q9UPT5. |

15. The method of preparing the cell-derived vesicle of claim 13, further comprising (2) delivering the cell-derived vesicle into a cell.
